(19) United States
(12) Patent Application Publication
(10) Pub. No.: US 2025/0295754 A1
(43) Pub. Date: Sep. 25, 2025

(54) Method for Obtaining SARS-CoV-2 Aerosol Variants for Oral Vaccines Inducing Gut-based Immunity Against COVID-19

(71) Applicant: Steven Mark Hayden, Reno, NV (US)

(72) Inventor: Steven Mark Hayden, Reno, NV (US)

(21) Appl. No.: 18/615,492

(22) Filed: Mar. 25, 2024

Related U.S. Application Data
(60) Provisional application No. 63/441,089, filed on Jan. 25, 2023.

(51) Int. Cl. A61K 39/215; A61K 39/00; A61K 39/12; A61K 39/145; A61P 31/14; G01N 1/22; G01N 1/40

(52) U.S. Cl. CPC A61K 39/215; A61K 39/12; A61K 39/145; A61P 31/14; G01N 1/22; G01N 1/4077; A61K 2039/525; A61K 2039/542; G01N 2001/2244; G01N 2001/4088

(57) ABSTRACT

A method involving collecting viral particles from exhaled breath for developing oral vaccines against diseases like COVID-19. Infected individuals exhale breath into sterile equipment to separate viral particles through centrifuging and filtering, excluding saliva and other pathogens. The process avoids heat or methods that could destroy the viruses, ensuring the aerosol's vitality. Purified extracts, suspended in cold saline or water, undergo optional screening for contaminants and multiplication in sterile conditions. The final product is a quantified, purified aerosol extract of the virus, used for oral vaccine development.

### FIELD OF THE INVENTION

The present invention relates generally to vaccine and gut-based immunity. More specifically, the present invention relates to oral vaccines comprising viral aerosol variants for inducing gut-based immunity against viral disease, more specifically with SARS-CoV-2 for mounting immunity against COVID-19 infection.

### BACKGROUND OF THE INVENTION

Coronaviruses are a diverse group of viruses infecting many different animals, and they can cause mild to severe respiratory infections in humans. In 2002 and 2012, respectively, two highly pathogenic coronaviruses with zoonotic origin, severe acute respiratory syndrome coronavirus (SARS-CoV) and Middle East respiratory syndrome coronavirus (MERS-CoV), emerged in humans and caused fatal respiratory illness, making emerging coronaviruses a new public health concern in the twenty-first century.

At the end of 2019, Severe acute respiratory syndrome coronavirus 2 (SARS-CoV-2) emerged as a highly transmissible and pathogenic coronavirus that emerged in late 2019 and caused a pandemic of acute respiratory disease, named ‘coronavirus disease 2019’ (COVID-19), which has caused major havoc to human health and public safety owing to being extremely transmissible, especially via the mode of exhalation from an infected COVID-19 subject to other people in the vicinity owing to various aerosolized viral samples of SARS-CoV-2 strains and has claimed numerous lives and destroyed many lives owing to long COVID-19 and other morbidities.

COVID-19 transmission via exhaled aerosol particles has been considered an important route for the spread of infection, especially during super-spreading. Patients who are positive for COVID-19 are known to exhale aerosol particles of SARS-CoV-2 that is known to be responsible for aerosol based and household transmission (Alsved M., et al., 2022).

Self-experimentation refers to when researchers have conducted experiments on themselves. Many self-experimentation studies are not published due to the lack of ethical approval, fear of employers' consequences or social punishment. The urgency to develop a vaccine for COVID-19 has refueled the tradition of self-experimentation and has sparked extensive discussion in the world of research ethics about the morality of self-experimentation. To date, the ‘Nuremberg Code’ is the only direct address to self-experimentation for medical ethics regulations. It is stated in Article 5 that, “No experiments should be conducted where there is a prior reason to believe that death or injury will occur; except, perhaps, in those experiments where experimental physicians also serve as subjects.”

The current vaccines for COVID-19 are mostly administered via the intramuscular route and they have many side effects as shown by Hamid M., 2022, Ahmed A., et al., 2023, Wharton B. R., et al., 2022, and less mucosal protection than swallowing a live virus. Live virus can be obtained from saliva, but saliva has potential blood, cells and other pathogens. Thus, a method of collection that can give best aerosol variant for live oral COVID-19 vaccine without the abovementioned side effects and need for extensive processing to get rid of potentially contaminating extraneous matter.

To address the aforementioned problems and needs, the present invention provides an alternate vaccine targeting SARS-CoV-2 strains via the mode of oral vaccination against COVID-19 by using aerosol SARS-CoV-2 strain based oral compositions for vaccines that can be administered to subjects and enhance immunity against COVID-19 by inducing gut-based immunity against COVID-19.

### SUMMARY OF THE INVENTION

The present invention provides oral vaccines targeting coronavirus disease 19 (COVID-19) as an alternative to current intramuscular vaccines, the oral vaccines comprising collecting severe acute respiratory syndrome coronavirus 2 (SARS-CoV-2) aerosol variants from a subject positive for COVID-19 to obtain a live SARS-CoV-2 anti-COVID-19 vaccine made from the collected SARS-CoV-2 that can be administered to orally to another subject to develop gut-based immunity against COVID-19. The present invention discloses and argues in favor of self-experimentation in trained physicians to overcome major and eminent challenges such as the COVID-19 pandemic to provide alternate and more accessible vaccines.

An aspect of the present invention relates to method of collection of viral particles causing a viral disease in aerosol form from exhaled breath, the method comprising the steps of: recruiting and selecting an infected human being/individual/subject exhaling viruses or viral particles; collecting exhaled breath comprising aerosol strain of a virus or viral particles from said infected human being/individual/subject in a collection equipment including a test tube or conical flask or a tube leading to a collection chamber, which is sterile, wherein the collection equipment may be coiled to spin out any saliva, other pathogens including bacteria, other extraneous matter; spinning or centrifuging the collection equipment to further spin out remaining saliva, other pathogens including bacteria, other extraneous matter and large particles; removing aerosol with remaining suspended smallest particles by decanting or via airstream separation or even using a plastic bag to obtain isolated aerosol comprising viral particles; passing the isolated aerosol comprising viral particles through further filters and filtration steps to obtain a purified aerosol extract with aerosol viral strains that are of micron size or alternatively or optionally collecting a purified aerosol extract with saline charged spray, wherein the passing step avoids heating, low humidity, and electromagnetic spectrum processes that kills aerosolized viral particles, wherein filter is food and water safe, wet and moist, and wherein the purified aerosol extract is collected with cold purified water while avoiding light or heat to maintain vitality of purified aerosol extract suspended in cold water or cold normal saline; optionally, screening the purified aerosol extract suspended in cold water or cold normal saline as obtained for other pathogens and toxins contamination to isolate screened and purified aerosol extract suspended in cold water or cold normal saline, and further optionally, multiply said screened and purified aerosol extract suspended in cold water using viral multiplication in sterile culture conditions to obtain multiplied, screened and purified aerosol extract suspended in cold water or cold normal saline; and quantifying purified aerosol extract suspended in cold water or cold normal saline, and/or screened and purified aerosol extract suspended in cold water or cold normal saline, and/or multiplied, screened and purified aerosol extract suspended in cold water or cold normal saline, comprising viral particles as selected above to obtain viral particles causing a viral disease in aerosol form for development of oral vaccines against said viral disease.

An aspect of the present invention relates to a method of collecting severe acute respiratory syndrome coronavirus 2 (SARS-CoV-2) aerosol variants, the method comprising the steps of: selecting a subject who is positive for coronavirus disease 19 (COVID-19); collecting an exhaled breath sample from said subject to obtain an exhaled breath sample; isolating an aerosolized SARS-CoV-2 variant sample from said exhaled breath sample; processing said aerosolized SARS-CoV-2 variant sample to obtain a processed aerosolized SARS-CoV-2 variant sample, wherein said processed aerosolized SARS-CoV-2 variant sample is collected for formation of an oral composition comprising said processed aerosolized SARS-CoV-2 variant sample to be administered as an oral vaccine against COVID-19, and wherein the subject is a human being.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 depicts an embodiment of the present invention that shows a simple method of exhaled breath collection from a sick, infected, COVID-19 positive subject, where the collected exhaled breath is processed using a filter of pore size of 200 nm or 0.2 μm.

FIG. 2 depicts an embodiment of the present invention that shows a method of exhaled breath collection from a sick, infected COVID-19 subject is positioned in a mobile laboratory or a similar stationary set up with their face in the opening of a metal funnel and asked to breathe normally, although exhaling through the mouth, for about 10 minutes to collect an exhaled breath sample from the breathing exercise.

FIG. 3 depicts an embodiment of the present invention that shows a pathogen air quality monitor that comprises a batch-type wet-wall glass cyclone or a wet cyclone coupled to a micro-immuno-electrode (MIE) detection unit that houses an automated liquid handling unit and MIE biosensor assembly, where the wet cyclone is connected to a high-flow vacuum pump to sample air.

FIG. 4 depicts an embodiment of the present invention illustrating developed gut-based T-cell immunity in stool samples without developing any lung infection or lung involvement.

FIG. 5 depicts an embodiment of the present invention illustrating stool samples PCR positive for COVID-19 causing SARS-CoV-2 exposure without developing any lung infection or lung involvement.

### DETAILED DESCRIPTION OF THE INVENTION

Detailed embodiments of the present invention are disclosed herein. However, it is to be understood that the disclosed embodiments are merely exemplary of the present invention, which may be embodied in various systems. Therefore, specific details disclosed herein are not to be interpreted as limiting, but rather as basis for teaching one skilled in the art to variously practice the present invention.

All illustrations of the drawings are for the purpose of describing selected versions of the present invention and are not intended to limit the scope of the present invention. The present invention may be understood more readily by reference to the following detailed description of the invention taken in connection with the accompanying drawing figures, which forms a part of this disclosure. It is to be understood that this invention is not limited to the specific devices, medicines, systems, conditions or parameters described and/or shown herein and that the terminology used herein is for the example only, and is not intended to be limiting of the claimed invention. Also, as used in the specification including the appended claims, the singular forms ‘a’, ‘an’, and ‘the’ include the plural, and references to a particular numerical value includes at least that particular value unless the content clearly directs otherwise. Ranges may be expressed herein as from ‘about’ or ‘approximately’ another particular value. When such a range is expressed, it is another embodiment. Also, it will be understood that unless otherwise indicated, dimensions and material characteristics stated herein are by way of example rather than limitation, and are for better understanding of sample embodiment of suitable utility, and variations outside of the stated values may also be within the scope of the invention depending upon the particular application.

The terms “comprising,” “having,” “including,” and “containing” are to be construed as open-ended terms (i.e., meaning “including, but not limited to,” and permit the presence of one or more features or components) unless otherwise noted. It should be understood that while various embodiments in the specification are presented using “comprising” language, under various circumstances, a related embodiment may also be described using “consisting of” or “consisting essentially of language.

As such, the terms “a” (or “an”), “one or more,” and “at least one” can be used interchangeably herein. Furthermore, “and/or” where used herein is to be taken as specific disclosure of each of the two specified features or components with or without the other. Further, unless specifically stated or obvious from context, as used herein, the term “or” is understood to be inclusive. Also, unless specifically stated or obvious from context, as used herein, the terms “a”, “an”, and “the” are understood to be singular or plural.

In any of the ranges described herein, the endpoints of the range are included in the range. However, the description also contemplates the same ranges in which the lower and/or the higher endpoint is excluded. Additional features and variations of the invention will be apparent to those skilled in tire art from the entirety of this application, including the drawing and detailed description, and all such features are intended as aspects of the invention. Likewise, features of the invention described herein can be re-combined into additional embodiments that also are intended as aspects of the invention, irrespective of whether the combination of features is specifically mentioned above as an aspect or embodiment of the invention. Also, only such limitations which are described herein as critical to the invention should be viewed as such; variations of the invention lacking limitations which have not been described herein as critical are intended as aspects of the invention.

Unless defined otherwise, all technical and scientific terms used herein have the same meaning as commonly understood by one of ordinary skill in the art to which this disclosure is related.

Units, prefixes, and symbols are denoted in their Systems International de Unites (SI) accepted form. Numeric ranges are inclusive of the numbers defining the range. The headings provided herein are not limitations of the various aspects or aspects of the disclosure, which can be had by reference to the specification as a whole. The entire document is intended to be viewed as a unified disclosure, and it should be understood that all combinations of features described herein are contemplated. All references cited herein are hereby incorporated by reference in their entireties.

As discussed hereinabove, there remains a problem and need for alternate and effective COVID-19 vaccines. The present invention provides such an alternative in the form of an oral anti-COVID-19 vaccine comprising live SARS-CoV-2 variants isolated and obtained from processing exhaled breath comprising aerosol live SARS-CoV-2 variants from sick, infected, COVID-19 positive individuals or subjects. The process as disclosed in the present invention eliminates saliva and most bacteria in directly sharing live SARS-CoV-2 responsible for COVID-19. The present invention, when used on a large scale would allow reaching the best and dominant aerosol variant and best multiplied variant from viral culture to be selected as live virus for inducing mucosal immunity against COVID-19 when the vaccine comprising the said live virus isolated initially from exhaled breath sample of COVID-19 positive patients is taken orally.

Additionally, the present invention presents a case in favor of self-experimentation by physicians, and contends that, if self-experimentation is recognized formally, more self-experimenters would seek oversight and review. The role of the ethics committee for the review of self-experimentation should be inspiring and persuasive, acting as a mirror for better self-reflection. Ethics committee oversight should be available for self-experimenters to a similar extent as any other formally organized research. The process should be inexpensive and simple so that self-experimenters feel empowered to access this facility. Without the threat of redundancy or social ostracism, self-experimentation may gain a spotlight with champions leading from the front rather than hiding their work and results. An attempt to ban self-experimentation is likely to be self-defeating and potentially encourage further secrecy.

The present invention provides a method of collecting COVID-19 aerosols for oral medicine to induce gut-based immunity. For the disclosed method of the present invention, otherwise healthy donors other than being COVID-19 without any mouth sores and adenopathy are selected if they are PCR-positive or antigen-positive for COVID-19 for collection of exhaled breath comprising aerosolized SARS-CoV-2. The best donors have highest aerosolized SARS-CoV-2 count and are rapidly positive on antigen testing. Using such a natural aerosol process as provided in the present invention, it allows isolation of most aerosolized and best variant of SARS-CoV-2 for oral vaccine development so as to induce COVID-19 immunity in vaccinated individuals or subjects.

Mucosal immunization has potential benefits over conventional parenteral immunization, eliciting immune defense in both mucosal and systemic tissue for protection from pathogen invasion at mucosal surfaces. To provide a first line of protection at these entry ports, mucosal vaccines have been developed and hold a significant promise for reducing the burden of infectious diseases. However, until very recently, only limited mucosal vaccines have been available. Li M., et al., 2020, which is incorporated herein by reference, provides appropriate administration routes, reasonable formulations, antigen-sampling and immune responses of mucosal immunity, and the strategies used to improve mucosal vaccine efficacy. Mucosal immunity and first public internet live virus inoculation using live virus is provided for reference on www.antivirusair.com and incorporated herein the present disclosure by reference.

The present invention provides methods of collecting SARS-CoV-2 aerosol variants which allows for selection of the best aerosolized and fastest reproducing virus for isolation live SARS-CoV-2 virus multiplication and use as a live oral virus to induce immunity with minimum lung complications.

The present invention discloses an improvement on what currently exists as no other known process allows for the selection of best aerosolized and fastest reproducing viral strains of SARS-CoV-2 for live virus multiplication and subsequent use as a live oral virus to induce mucosal immunity with minimum lung complications. In contrast, the intramuscular vaccines are not known to create any mucosal immunity and instead present with complications and side effects such as excessive autoimmune side effects. Many in the society think that using saliva as a source of live SARS-Cov-2 strains/variants that cause COVID-19 is gross and dirty. However, saliva from healthy donors, who have no acute sickness, no sores, no adenopathy is exceptionally healthy.

The present invention presents a method that eliminates saliva and most bacteria in directly sharing live SARS-CoV-2 strains/variants causing COVID-19. The large particles containing spit, saliva, and potential pathogens are literally spun out of aerosol form as in exhaled breath, while the smaller particles such as aerosolized virus or viral particles having a higher drag to mass ratio remain in aerosol form for a longer time and distance from point of exhalation. Such a difference allows for differential collection of viruses or viral particles such as COVID-19 causing SARS-CoV-2 aerosol variants/strains and such a methodology is exploited, developed, disclosed, and used in the present invention. Further, it is contended that such a process as disclosed herein, when used on a large scale allows collection, isolation, and selection of best aerosol variant and best multiplied variant of COVID-19 causing, SARS-CoV-2 virus. It is proposed that large institutions and government organizations can do viral multiplication using such viral cultures as disclosed in the method of the present invention. The viral multiplication steps as disclosed herein can further preferentially select the most rapidly reproducing variants of SARS-CoV-2. Poor physicians can also similarly use the method as discloses as easily and economically by spinning out larger spit mucous and bacterial products from the exhaled breath collected from a COVID-19 positive individual/subject without needing expensive resources.

Also, the method of the present invention can be used to collect unknown aerosols beyond SARS-CoV-2 and COVID-19, collecting other aerosolized viruses for similar gut- and mucosal-immunity inducing live inoculation via oral route. It may for instance work for aerosolized viruses such as RSV and influenza.

Embodiments will now be described in details with reference to the accompanying drawings. To avoid unnecessarily obscuring in the present disclosure, well-known features may not be described, or substantially the same elements may not be redundantly described, for example. This is for ease of understanding. The drawings and the following description are provided to enable those skilled in the art to fully understand the present disclosure and are in no way intended to limit the scope of the present disclosure as set forth in the appended claims.

An embodiment of the present invention provides a method of collection of viral particles causing a viral disease in aerosol form from exhaled breath, the method comprising the steps of: recruiting and selecting an infected human being/individual/subject exhaling viruses or viral particles; collecting exhaled breath comprising aerosol strain of a virus or viral particles from said infected human being/individual/subject in a collection equipment including a test tube or conical flask or a tube leading to a collection chamber, which is sterile, wherein the collection equipment may be coiled to spin out any saliva, other pathogens including bacteria, other extraneous matter; spinning or centrifuging the collection equipment to further spin out remaining saliva, other pathogens including bacteria, other extraneous matter and large particles; removing aerosol with remaining suspended smallest particles by decanting or via airstream separation or even using a plastic bag to obtain isolated aerosol comprising viral particles; passing the isolated aerosol comprising viral particles through further filters and filtration steps to obtain a purified aerosol extract with aerosol viral strains that are of micron size or alternatively or optionally collecting a purified aerosol extract with saline charged spray, wherein the passing step avoids heating, low humidity, and electromagnetic spectrum processes that kills aerosolized viral particles, wherein filter is food and water safe, wet and moist, and wherein the purified aerosol extract is collected with cold purified water while avoiding light or heat to maintain vitality of purified aerosol extract suspended in cold water or cold normal saline; optionally, screening the purified aerosol extract suspended in cold water or cold normal saline as obtained for other pathogens and toxins contamination to isolate screened and purified aerosol extract suspended in cold water or cold normal saline, and further optionally, multiply said screened and purified aerosol extract suspended in cold water using viral multiplication in sterile culture conditions to obtain multiplied, screened and purified aerosol extract suspended in cold water or cold normal saline; and quantifying purified aerosol extract suspended in cold water or cold normal saline, and/or screened and purified aerosol extract suspended in cold water or cold normal saline, and/or multiplied, screened and purified aerosol extract suspended in cold water or cold normal saline, comprising viral particles as selected above to obtain viral particles causing a viral disease in aerosol form for development of oral vaccines against said viral disease.

Another embodiment of the method of collection of viral particles causing a viral disease in aerosol form from exhaled breath as disclosed herein the present invention, wherein the viral disease includes diseases caused by Respiratory syncytial virus (RSV), influenza virus.

Another embodiment of the method of collection of viral particles causing a viral disease in aerosol form from exhaled breath as disclosed herein the present invention, where it is mass produced from a single processor, great security and quality assurance are to be tightly followed along with testing and assurance compliance. On the other hand, physicians who use the method as disclosed and are not able to afford aforementioned expensive and elaborate quality assurance methods, must still screen all donors of exhaled breath for mouth sores, adenopathy, and exclude those donors right away before further screening and testing for other pathogens and toxins contamination who are deemed unhealthy for exhaled breath donation. It must be ensured that the collected purified viral particles in aerosol method as disclosed herein are kept viable and alive form in cold water or cold normal saline conditions, sterile environment without harmful exposure to heat or light or optionally, if necessary, in suspended animation in a lyophilized form if used by larger institutions who can economically afford this alternative. The isolated, purified aerosolized spun viral variants as aforesaid can be cultured under proper sterile growth media conditions to allow viral replication that allows for variants with highest reproductive rate to be selectively chosen. Similarly, large scale production must be accompanied by quality control including screening for viability and count of the viruses, along with a check for absence of contamination by other pathogens such as bacteria, other viruses, and toxin in the cold water or cold normal saline containing the purified and screened viral particles or viral strains as collected from the method as disclosed hereinabove. The advantage of using cold saline preferably for suspending collected viral particles in the method herein disclosed is that in the subsequent method of oral vaccination, it reminds the vaccinating individuals/subjects/consumers, used interchangeably, to not to consume the offered oral vaccine in cold normal saline by binge drinking and rather to pace themselves. Also, the collected viral particles causing a disease in aerosol form from exhaled breath should be properly stored under sterile and safe storage space with proper labelling until consumed by desired individuals/subjects/consumers such as the ones at risk of breathing aerosol containing viruses such as exposure by air borne transmission of viral diseases.

An embodiment of the present invention provides a method of producing an oral vaccine against a viral disease, the method comprising the steps of: collecting, purifying, and selecting viral particles causing a viral disease in aerosol form from exhaled breath as disclosed herein the present invention; quantifying the amount of quantity of suspended viral particles causing a disease in aerosol form from exhaled breath; preparing a drink of cold water or cold normal saline under sterile conditions for consumption by a consumer as an oral vaccine, wherein the drink of cold water or cold normal saline is in a range of 1 to 15 cc quantity, and wherein the drink of cold water or cold normal saline comprises a pre-quantified amount of quantity of suspended viral particles causing a disease in aerosol form from exhaled breath.

Another embodiment of the present invention providing the method of producing an oral vaccine against a viral disease as disclosed hereinabove, wherein the oral vaccine induces gut-based mucosal immunity against viral diseases.

Another embodiment of the present invention providing an oral vaccine against a viral disease, the oral vaccine comprising viral particles causing a viral disease in aerosol form from exhaled breath as disclosed herein the present invention; and cold water or cold normal saline under sterile conditions.

An embodiment of the present invention provides a method of collecting severe acute respiratory syndrome coronavirus 2 (SARS-CoV-2) aerosol variants, the method comprising the steps of: selecting a subject who is positive for coronavirus disease 19 (COVID-19); collecting an exhaled breath sample from said subject to obtain an exhaled breath sample; isolating an aerosolized SARS-CoV-2 variant sample from said exhaled breath sample; processing said aerosolized SARS-CoV-2 variant sample to obtain a processed aerosolized SARS-CoV-2 variant sample, wherein said processed aerosolized SARS-CoV-2 variant sample is collected for formation of an oral composition comprising said processed aerosolized SARS-CoV-2 variant sample to be administered as an oral vaccine against COVID-19, and wherein the subject is a human being.

Another embodiment of the present invention provides the method of collecting severe acute respiratory syndrome coronavirus 2 (SARS-CoV-2) aerosol variants as disclosed herein, wherein the collecting an exhaled breath sample is done in a collection equipment including a test tube or conical flask or a tube leading to a collection chamber, which is sterile, and wherein the collection equipment may be coiled to spin out any saliva, other pathogens including bacteria, other extraneous matter.

Another embodiment of the present invention provides the method of collecting severe acute respiratory syndrome coronavirus 2 (SARS-CoV-2) aerosol variants as disclosed herein, wherein the isolating an aerosolized SARS-CoV-2 variant sample is done by spinning or centrifuging the collection equipment to further spin out remaining saliva, other pathogens including bacteria, other extraneous matter and large particles; removing aerosol with remaining suspended smallest particles by decanting or via airstream separation or even using a plastic bag to obtain the aerosolized SARS-CoV-2 variant sample.

Another embodiment of the present invention provides the method of collecting severe acute respiratory syndrome coronavirus 2 (SARS-CoV-2) aerosol variants as disclosed herein, wherein the processing said aerosolized SARS-CoV-2 variant sample involves passing the isolated aerosol comprising viral particles through filters and filtration steps to obtain the processed aerosolized SARS-CoV-2 variant sample, wherein the filters that are of micron size, wherein the passing step avoids heating, low humidity, and electromagnetic spectrum processes that kills aerosolized viral particles, wherein filter is food and water safe, wet and moist, and wherein the processed aerosolized SARS-CoV-2 variant sample is further collected with cold purified water while avoiding light or heat to maintain vitality. Another embodiment of the present invention provides the method of collecting severe acute respiratory syndrome coronavirus 2 (SARS-CoV-2) aerosol variants as disclosed herein, wherein the processing said aerosolized SARS-CoV-2 variant sample involves alternatively or optionally collecting a purified aerosol extract with saline charged spray to obtain the processed aerosolized SARS-CoV-2 variant sample, wherein the collecting with saline charged spray avoids heating, low humidity, and electromagnetic spectrum processes that kills aerosolized viral particles, and wherein the processed aerosolized SARS-CoV-2 variant sample is further collected with cold purified water while avoiding light or heat to maintain vitality.

An embodiment of the present invention provides a method of producing an oral vaccine against coronavirus disease 19 (COVID-19), the method comprising the steps of: obtaining a processed aerosolized severe acute respiratory syndrome coronavirus 2 (SARS-CoV-2) variant sample by a method as disclosed herein the present invention; purifying the processed aerosolized SARS-CoV-2 variant sample by passing it through filters to remove any remaining extraneous matter and contaminants and suspending the filtered sample in cold water or cold normal saline under sterile conditions to obtain purified and suspended aerosolized SARS-CoV-2 variant sample; quantifying the amount of quantity of purified and suspended aerosolized SARS-CoV-2 variant sample; and preparing a drink of cold water or cold normal saline under sterile conditions for consumption by a consumer as an oral vaccine, wherein the drink of cold water or cold normal saline is in a range of 1 to 15 cc quantity, wherein the drink of cold water or cold normal saline comprises a pre-quantified amount of quantity of purified and suspended aerosolized SARS-CoV-2 variant sample.

Another embodiment of the present invention provides the method of producing an oral vaccine against coronavirus disease 19 (COVID-19) as disclosed herein, wherein the oral vaccine induces gut-based mucosal immunity against SARS-CoV-2 caused COVID-19. Another embodiment of the present invention providing an oral vaccine against viral diseases, the oral vaccine comprising processed aerosolized severe acute respiratory syndrome coronavirus 2 (SARS-CoV-2) variant sample by a method as disclosed herein the present invention; and cold water or cold normal saline under sterile conditions.

The spinning, filtration, and purification steps of aerosol viral strain collection removes large spit particles, majority of bacteria, toxins, and contaminants. The best aerosol variants or strains of SARS-CoV-2 are always those with the smallest particle size. The best aerosolized COVID-19 variant has higher mass to drag ratio and resists being spun out of the aerosol volume and remains suspended. The selection of small particle size preferentially selects the best aerosolized variant. In large scale production of purified and suspended aerosolized SARS-CoV-2 variant sample in cold water or cold normal saline, the use of viral culture media preferentially selects the most rapidly multiplying viral particles.

The centrifugal force of the coiled tube and spinning collection bag removes larger particles as spit, bacteria, toxins and contaminants. In the final purified and suspended aerosolized SARS-CoV-2 variant sample in cold water or cold normal saline, the trace amounts of bacteria and other extraneous matter are removed with filtration steps by trapping such larger particles while allowing smaller viral particles through.

For the invention as disclosed herein, a preferred embodiment comprises the use of a coiled tube leading to a plastic bag, which is spun to further separate larger particles containing spit, bacteria, other pathogens, other contaminants, and toxins, etc. from the aerosol form of the SARS-CoV-2 or other viral variants. All elements which provide centrifugal force to remove bacteria and other extraneous matter are essential. Regardless of how the method as disclosed herein the present invention is used, centrifugal force upon exhaled breath from the sick, infected COVID-19 positive subject/individual/patient/donor, used interchangeably, is necessary to remove all other larger particles.

The reason for these processing, isolation, and purification steps arises from the hesitation of consumers as well as dislike for consuming other's spit and potential problems with contaminants, and pathogens such as bacteria in the exhaled breath from donors. In contrast, people may be willing to consume and swallow an oral vaccine even if originates from exhaled breath containing spit, bacteria, etc, and contains live SARS-CoV-2 aerosol variants from a donor. Central processing and viral culture multiplication allows centralized quality control of product sealing product and checking for toxins and other pathogens. It is extremely important that central processing has rigorous testing for quality and elimination of other toxins and pathogens. SARS-CoV-2 culture and multiplication may monitor the gene sequence of the best aerosolized variant and which variant multiplies the fastest in viral culture. The method can work even with the use of a simple coiled tube leading to an expanding plastic bag and use a washable coffee filter N95 mask or HEPA filter to collect.

Coronavirus has existed in the guts of animals and caused virtually no deaths, only a little diarrhea for thousands of years. Everyone is exposed to viruses by eating them by the tens of thousands every day but rarely any viral infection is noticed. A virus usually cannot enter a host's cell to reproduce without going through a viral receptor doorway to enter the cell. However, as the coronavirus became aerosolized, humans developed lung breathing complications along with disease, dysfunction, and discomfort. Luckily, very few viruses are well aerosolized. Generally, human beings and other animal guts have a viral immune system that has gut viral receptors to receive the virus and process it in the guts. It is tested in the present invention that one can develop gut immunity by oral exposure and it suppresses respiratory transmission. Gut immunity to coronavirus can only be acquired by exposure of guts to live virus and the same is tested in the present invention as discussed hereinbelow. Initially all coronavirus research was restricted to CDC level 3 Biohazard labs, but in 2020, the inventor-physician operated a PCR collection site for Covid-19 positivity testing. So, samples from COVID-19 positive donors of SARS-CoV-2 via donations were received and used for experimentation as disclosed in the present invention in the Examples below. The present invention further provides that the key to stopping exhaled transmission of coronavirus such as SARS-CoV-2 is gut exposure to live coronavirus as SARS-CoV-2 to develop gut-based immunity.

The invention will be further explained by the following Examples, which are intended to purely exemplary of the invention, and should not be considered as limiting the invention in any way.

### EXAMPLES

### Example 1: Collection and Isolation of Sars-Cov-2 Aerosol Variants

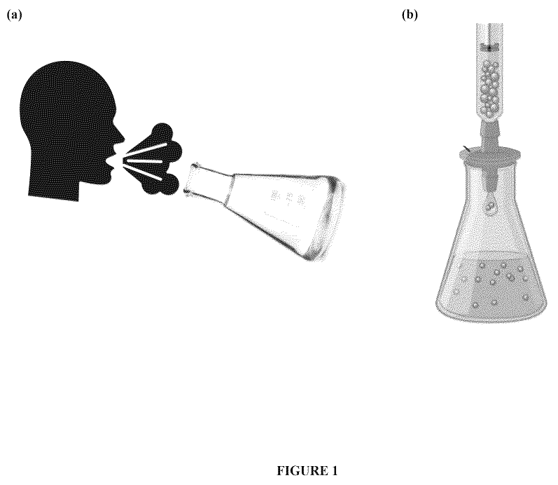

In this example, isolation, culture and identification of severe acute respiratory syndrome coronavirus 2 (SARS-CoV-2) aerosol variants is provided as a representative method of the present invention. Firstly, a sample of breath is collected from a sick, COVID-19 positive subject. A selection criterion for the sick, COVID-19 positive subject takes into account the collection of breath from patients exhaling SARS-CoV-2-containing aerosol particles close to symptom onset who are more likely to transmit COVID-19 as deduced in Alsved M. et al., 2022, which is incorporated here by reference. The breath sample is processed to remove all extraneous matter other than SARS-CoV-2 variant to isolate an aerosolized SARS-CoV-2 variant sample. The sample is collected under sterile conditions from as close as possible from mouth of potentially or known COVID-19 positive or infected person. The breath donor can put lips on tube and exhale directly into tube. FIG. 1 provides a simple iteration of an embodiment of the present invention providing a method of exhaled breath sample collection, where a sick, COVID-19 positive subject is asked to breath in a modified test tube-based device and the exhaled breath sample containing SARS-CoV-2 aerosol variant sample is collected for further processing and isolation of an aerosolized SARS-CoV-2 variant sample.

For the aforementioned processing, the breath sample can pass through a dust cyclone filter to remove most of the dust larger than 1 micron. The breath sample can be passed through a washable HEPA filter. The washable HEPA filter has a continuous stream of water running through it, rinsing it with SARS-CoV-2 viral growth media. The SARS-CoV-2 viral growth media passing through the filter is then filtered with a known viral filter to extract SARS-CoV-2 variants. The temperature is kept ideal for SARS-CoV-2 viral growth. The SARS-CoV-2 virus variants collected from growth media can be concentrated by known viral filters, or the SARS-CoV-2 virus variants can be multiplied by culture and then extracted. Using the alternate breath collection process as described above, which are used for public health projects, samples of the SARS-CoV-2 viral collections are measured by the Public Health Department lab to evaluate the SARS-CoV-2 mutant strain and evaluate any bacterial or chemical pollutants, and they can be similarly processed to obtain other isolated SARS-CoV-2 strain samples. The isolation process follows standard known protocols and processes as have been shown in https://bio.libretexts.org/Courses/Mansfield_University_of_Pennsylvania/BSC_3271%3A_Microbiology_for_Health_Sciences_Sp21_(Kagle)/03%3A_Viruses/3.01%3A_Viral_Replication/3.1.03%3A_Isolation_Culture_and_Identification_of_Viruses, which is incorporated here by reference.

An alternative is to place the collection tube about 6 inches overhead of crowd to collect samples in crowded areas. Here, breath can be collected from public places as is usually done for public health monitoring from public transport areas where people gather to collect viral samples. Similarly, at clinics, where COVID-19 sick people are gathered for treatments, samples can be collected alongside assessment of dominant mutant from the aerosol samples that is causing sickness at a particular time. In a similar manner as above, the breath samples are processed to remove all extraneous matter to isolate other aerosolized SARS-CoV-2 variant samples.

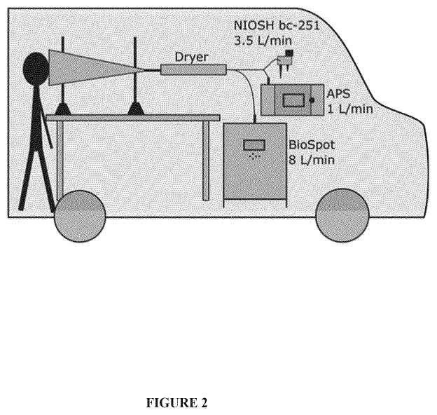

Alsved M. et al., 2022, provides another alternate methodology for aerosol sample collection using cyclone method as shown in FIG. 2 which provides the process of exhaled breath collection from a sick, infected COVID-19 positive subject showing symptoms. FIG. 2 illustrates one of the methods where, said COVID-19 positive subject is positioned in a mobile laboratory or a similar stationary set up with their face in the opening of a metal funnel and asked to breathe normally, although exhaling through the mouth, for about 10 minutes to collect an exhaled breath sample from the breathing exercise.

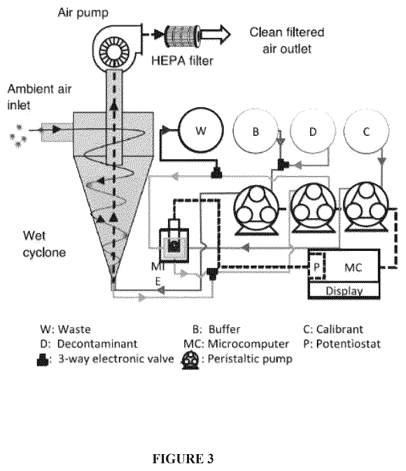

Another such alternate method for SARS-CoV-2 aerosol detection and collection is based on the work of Puthussery J. V., et al., 2023, which is illustrated in FIG. 3, showing a schematic showing a wet cyclone particle into liquid samplers (PILS) coupled with a MIE detection unit comprising a submerged micro-immuno-electrode (MIE) biosensor connected to a potentiostat and automated liquid handling accessories, for the method of breath collection from a public place to isolate and obtain an alternate aerosolized SARS-CoV-2 variant sample, which is incorporated here by reference.

### Example 2: Self-Exposure to Collected Sars-Cov-2 Aerosol Variant in Form of an Oral Vaccine by Physicians

In this example, inventor who is also a physician, self-exposed himself to collected aerosolized SARS-CoV-2 variant sample that was suspended in cold normal saline in a volume of up to 15 cc and consumed by oral route by slow and controlled swallowing to expose the consumers' gut to develop induced gut-based mucosal immunity against the aerosolized SARS-CoV-2 variant in accordance with Nurenberg code. The oral live SARS-CoV-2 virus exposure acted as an oral vaccine.

WHO's statement of food contaminated with SARS-CoV-2 that otherwise may cause COVID-19 if transmitted via inhalation or in aerosol form is considered generally safe when exposure is via the oral route. The exposure is done with an understanding that gut has ACE2 receptors and that classic coronavirus infections cause gastrointestinal infections. Classically, gut coronavirus infections are known not to be deadly, producing only mild diarrhoea and cramps. Coronavirus gut infections produced natural immunity for centuries without pulmonary complications. The exception is overcrowded chickens kicking coronavirus into the air inhaling it and causing a lung infection. It is well known that gut coronavirus exposure caused natural mucosal immunity.

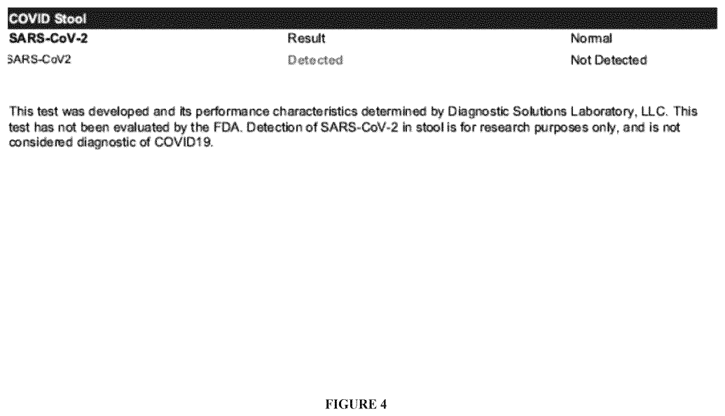

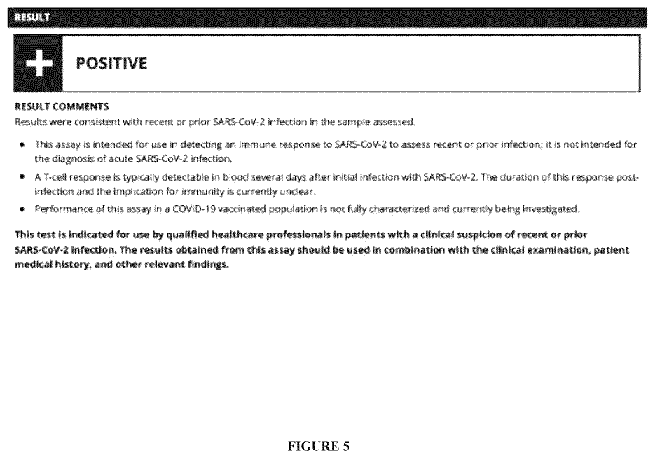

The consumer, physician-inventor procured SARS-CoV-2 donations from sick, infected, COVID-19 positive donors from a COVID-19-antigen testing facility. It was observed that after swallowing live aerosolized SARS-CoV-2 variant sample as an oral vaccine in cold water or cold normal saline well before secondary exposure by inhalation, the physician-inventors developed gut-based T-cell immunity without developing any lung infection or lung involvement at all as shown in FIG. 4. A video of the swallowing can be seen at: https://www.youtube.com/watch?v=6C45DZuizKY&t=2s. Further, it was observed that there was a small rise in high-sensitivity C-reactive protein (CRP), while the complete blood count (CBC), and comprehensive metabolic panel (CMP) remained virtually unchanged. There was no fever just tiredness post-consumption after the oral exposure and a single brief episode of loose but formed stool. The stool sample upon tests was positive by PCR testing for COVID-19 causing SARS-CoV-2 as shown in FIG. 5, however, interestingly and notably, the serum antibody titer of SARS-CoV-2 antigen count did not rise abnormally. It was notable because, initially it was widely but improperly believed that without major elevations of serum antibodies, anti-COVID-19 immunity could not be achieved. However, it is countered by gut-induced mucosal immunity that causes T-cell based defences to mount without “abnormal” levels of antibodies to spike protein of SARS-CoV-2 that causes COVID-19. This is shown with T-cell immunity mounting up without elevation of antibodies to spike protein seen in this example as well. The physician-inventor was exposed to live aerosolized SARS-CoV-2 variant sample by multiple, intermittent swallowing via the oral route and each exposure led to fewer symptoms and ultimately no more symptoms as shown on https://rumble.com/vlcbsu5-never-inhale-covid-inhalation-is-not-correct-path-to-immunity.html. The loose stool stopped after the first oral exposure dose itself. Thus, the self-experimentation experiment in this example showed proven T-cell immunity despite normal serum antibodies i.e., no antibody against COVID-19 causing SARS-CoV-2. This scheme of exposure thus stops transmission unlike conventional vaccine intramuscular exposure because they do not activate the gut-based immune system.

Follow up inhalation by aerosol or air-borne transmission and SARS-CoV-2 exposure thus proved protective as proven immunity through positive stool PCR was tested for immunity against inhalation exposure and did not turn PCR or antigen positive as shown in this representative example of the present invention. Also, there was no pulmonary COVID-19 infection or any sickness or symptoms. This was owing to the developed gut-based mucosal immunity against COVID-19 causing SARS-CoV-2 owing to repeat oral exposures to live aerosolized SARS-CoV-2 variant sample by swallowing it as an oral vaccine.

It will be apparent to those skilled in the art that various modifications and variations can be made in the practice of the present invention without departing from the scope or spirit of the invention. Other embodiments of the invention will be apparent to those skilled in the art from considering of the specification and practice of the invention. It is intended that the specification and examples be considered as exemplary only, with a true scope and spirit of the invention being indicated by the following claims.

What is claimed is:

1. A method of collection of viral particles causing a viral disease in aerosol form from exhaled breath, the method comprising the steps of:
recruiting and selecting an infected human being/individual/subject exhaling viruses or viral particles;
collecting exhaled breath comprising aerosol strain of a virus or viral particles from said infected human being/individual/subject in a collection equipment including a test tube or conical flask or a tube leading to a collection chamber, which is sterile, wherein the collection equipment may be coiled to spin out any saliva, other pathogens including bacteria, other extraneous matter;
spinning or centrifuging the collection equipment to further spin out remaining saliva, other pathogens including bacteria, other extraneous matter and large particles; removing aerosol with remaining suspended smallest particles by decanting or via airstream separation or even using a plastic bag to obtain isolated aerosol comprising viral particles;
passing the isolated aerosol comprising viral particles through further filters and filtration steps to obtain a purified aerosol extract with aerosol viral strains that are of micron size or alternatively or optionally collecting a purified aerosol extract with saline charged spray, wherein the passing step avoids heating, low humidity, and electromagnetic spectrum processes that kills aerosolized viral particles, wherein filter is food and water safe, wet and moist, and wherein the purified aerosol extract is collected with cold purified water while avoiding light or heat to maintain vitality of purified aerosol extract suspended in cold water or cold normal saline;
optionally, screening the purified aerosol extract suspended in cold water or cold normal saline as obtained for other pathogens and toxins contamination to isolate screened and purified aerosol extract suspended in cold water or cold normal saline, and further optionally, multiply said screened and purified aerosol extract suspended in cold water using viral multiplication in sterile culture conditions to obtain multiplied, screened and purified aerosol extract suspended in cold water or cold normal saline; and
quantifying purified aerosol extract suspended in cold water or cold normal saline, and/or screened and purified aerosol extract suspended in cold water or cold normal saline, and/or multiplied, screened and purified aerosol extract suspended in cold water or cold normal saline, comprising viral particles as selected above to obtain viral particles causing a viral disease in aerosol form for development of oral vaccines against said viral disease.

2. The method of claim 1, wherein the viral disease includes diseases caused by Respiratory syncytial virus (RSV), influenza virus.

3. The method, as claimed in claim 1, further comprising the steps of:
collecting, purifying, and selecting viral particles causing a viral disease in aerosol form from exhaled breath of claim 1;
quantifying the amount of quantity of suspended viral particles causing a viral disease in aerosol form from exhaled breath;
preparing a drink of cold water or cold normal saline under sterile conditions for consumption by a consumer,
wherein the drink of cold water or cold normal saline is in a range of 1 to 15 cc quantity, and
wherein the drink of cold water or cold normal saline comprises a pre-quantified amount of quantity of suspended viral particles causing a disease in aerosol form from exhaled breath.

4. The method as claimed in claim 3, wherein the oral vaccine induces gut-based mucosal immunity against viral diseases.

5. The method as claimed in claim 3, comprising an oral vaccine against a viral disease, the oral vaccine comprising viral particles causing a viral disease in aerosol form from exhaled breath obtained by method of claim 3; and cold water or cold normal saline under sterile conditions.

6. A method of collection of viral particles comprising the steps of:
selecting a subject who is positive for coronavirus disease 19 (COVID-19); collecting an exhaled breath sample from said subject to obtain an exhaled breath sample;
isolating an aerosolized SARS-CoV-2 variant sample from said exhaled breath sample;
processing said aerosolized SARS-CoV-2 variant sample to obtain a processed aerosolized SARS-CoV-2 variant sample,
wherein said processed aerosolized SARS-CoV-2 variant sample is collected for formation of an oral composition comprising said processed aerosolized SARS-CoV-2 variant sample to be administered as an oral vaccine against COVID-19, and wherein the subject is a human being.

7. The method as claimed in claim 6, wherein the collecting an exhaled breath sample is done in a collection equipment including a test tube or conical flask or a tube leading to a collection chamber, which is sterile, and wherein the collection equipment may be coiled to spin out any saliva, other pathogens including bacteria, other extraneous matter.

8. The method as claimed in claim 6, wherein the isolating an aerosolized SARS-CoV-2 variant sample is done by spinning or centrifuging the collection equipment to further spin out remaining saliva, other pathogens including bacteria, other extraneous matter and large particles; removing aerosol with remaining suspended smallest particles by decanting or via airstream separation or even using a plastic bag to obtain the aerosolized SARS-CoV-2 variant sample.

9. The method as claimed in claim 6, wherein the processing said aerosolized SARS-CoV-2 variant sample involves passing the isolated aerosol comprising viral particles through filters and filtration steps to obtain the processed aerosolized SARS-CoV-2 variant sample, wherein the filters that are of micron size, wherein the passing step avoids heating, low humidity, and electromagnetic spectrum processes that kills aerosolized viral particles, wherein filter is food and water safe, wet and moist, and wherein the processed aerosolized SARS-CoV-2 variant sample is further collected with cold purified water while avoiding light or heat to maintain vitality.

10. The method as claimed in claim 6, wherein the processing said aerosolized SARS-CoV-2 variant sample involves alternatively or optionally collecting a purified aerosol extract with saline charged spray to obtain the processed aerosolized SARS-CoV-2 variant sample, wherein the collecting with saline charged spray avoids heating, low humidity, and electromagnetic spectrum processes that kills aerosolized viral particles, and wherein the processed aerosolized SARS-CoV-2 variant sample is further collected with cold purified water while avoiding light or heat to maintain vitality.

11. The method of collection of viral particles, as claimed in claim 6, further comprising the steps of:
obtaining a processed aerosolized severe acute respiratory syndrome coronavirus 2 (SARS-CoV-2) variant sample by the method of claim 6;
purifying the processed aerosolized SARS-CoV-2 variant sample by passing it through filters to remove any remaining extraneous matter and contaminants and suspending the filtered sample in cold water or cold normal saline under sterile conditions to obtain purified and suspended aerosolized SARS-CoV-2 variant sample;
quantifying the amount of quantity of purified and suspended aerosolized SARS-CoV-2 variant sample; and
preparing a drink of cold water or cold normal saline under sterile conditions for consumption by a consumer as an oral vaccine,
wherein the drink of cold water or cold normal saline is in a range of 1 to 15 cc quantity, wherein the drink of cold water or cold normal saline comprises a pre-quantified amount of quantity of purified and suspended aerosolized SARS-CoV-2 variant sample.

12. The method as claimed in claim 11, wherein the oral vaccine induces gut-based mucosal immunity against SARS-CoV-2 caused COVID-19.

13. The method as claimed in claim 11, comprising an oral vaccine against viral diseases, the oral vaccine comprising: processed aerosolized severe acute respiratory syndrome coronavirus 2 (SARS-CoV-2) variant sample obtained by a method of claim 11; and cold water or cold normal saline under sterile conditions.
